(19) United States
(12) Patent Application Publication
(10) Pub. No.: US 2026/0283447 A1
(43) Pub. Date: Sep. 24, 2026

(54) NOVEL SUCTION VALVE

(71) Applicant: GA HEALTH COMPANY LIMITED, Hong Kong (CN)

(72) Inventors: Ken MCCABE, Hong Kong (CN); Tsan Ho CHAN, Hong Kong (CN)

(21) Appl. No.: 19/573,158

(22) Filed: Mar. 20, 2026

Related U.S. Application Data
(60) Provisional application No. 63/774,962, filed on Mar. 20, 2025.

(51) Int. Cl. A61B 1/00

(52) U.S. Cl. CPC A61B 1/00068; A61B 1/00042; A61B 1/0011

(57) ABSTRACT

The present invention is related to a suction valve, adaptable to position in a port of an endoscope for assisting a cleaning procedure for said endoscope, comprising: a main body, comprising an internal cavity; a shaft, comprising a tubular structure being made of a first material selected from thermosetting material, resilient material, or a combination thereof and comprising a first opening on the tubular wall of said tubular structure and a second opening at the bottom of said tubular structure, wherein said first opening and said second opening being fluidic communicable defining an internal channel inside said tubular structure; a button; a resilient member, positioned between said button and said main body, said main body, said shaft, said button, and said resilient member being assembled in a way such that said button together with said shaft being configured to reciprocally move between a first position where said first opening of said shaft being at least partially aligned with a first opening of said port and a second position where said first opening of said shaft being misaligned with said first opening of said port.

### FIELD OF THE INVENTION

The present invention is related to an endoscopic valve, in particular a suction valve, a specialized device designed for endoscopic procedures.

### BACKGROUND ART

In an endoscope, a suction valve is a critical manual control component, typically located on the control handle, that allows medical personnel to manage the aspiration of fluids and debris from the patient's body. This valve acts as a mechanical gate between the endoscope's internal suction channel and an external vacuum source. By pressing and releasing this valve, the medical personnel regulates the flow of air and liquid, ensuring a clear field of vision and the removal of obstructive materials during a procedure.

The primary use of the suction valve is to facilitate the evacuation of waste, such as blood, mucus, or digestive fluids, which can obscure the camera lens or interfere with the diagnostic process. When the medical personnel depresses the valve, it aligns an internal port with the suction channel, creating a continuous vacuum path that draws substances through the distal tip of the endoscope and into a collection canister. Beyond fluid removal, the valve is also utilized to collapse organs or reduce insufflated air when the procedure is nearing completion.

A state-of-the-art suction valve is a precision-engineered assembly characterized by a metal shaft, usually fabricated from medical-grade stainless steel or a similar high-performance alloy. This metal shaft serves as the structural core of the valve, providing the necessary rigidity to withstand repeated mechanical cycles and ensuring a tight, reliable fit within the valve cylinder.

Because a state-of-the-art suction valve must prevent air leaks, the clearance between the rigid metal shaft and the internal walls of the valve cylinder is exceptionally narrow. The tight mechanical tolerances, required to maintain a vacuum seal, deteriorates the aforementioned jamming problem. When the endoscope aspirates particulate matter—such as fibrous food debris or bone fragments—these hard particles can become wedged in this microscopic gap. The rigid nature of the metal shaft means it cannot deform to bypass the obstruction; instead, the debris creates an immediate mechanical interlock. This friction-based seize often occurs mid-stroke, leaving the valve stuck in a depressed position which causes continuous, uncontrolled suction that can potentially harm patient tissue.

Beyond the risk of sudden jamming, the use of the metal shaft introduces a significant long-term disadvantage regarding mechanical wear and material fatigue. Because the metal shaft is significantly harder than the internal housings or guide rails of the endoscope—which are often made of softer alloys or high-performance polymers—the repeated sliding motion during every procedure acts as a slow abrasive process. Over time, this interaction leads to the wearing or galling of the internal surfaces of the endoscope body. This wearing creates uneven grooves and metal-on-metal micro-shavings that further degrade the smooth operation of the valve. As this wear progresses, the structural integrity of the seal is compromised, leading to vacuum bypass and fluid leakage. Once the internal structure of the endoscope is sufficiently worn by the metal shaft, the valve may no longer seat correctly even if the shaft itself is replaced. This creates a permanent loss of suction efficiency and increases the risk of cross-contamination, as fluids can seep into the worn recesses of the device. Ultimately, this characteristic wear necessitates expensive and frequent overhauls of the entire endoscope handle, as the damage to the internal housing caused by the hard metal shaft is often irreversible without major industrial refurbishment.

During the course of an endoscopic operation, the suction valve is susceptible to mechanical failure if it becomes jammed or stuck by biological debris. This occurs when solid matter—such as fruit seeds, food particles, or bone fragments—is drawn into the suction channel and becomes lodged between the metal shaft and the inner wall of the valve housing. If the jammed valve is a modular, removable component, the medical personnel can simply extract the obstructed unit and replace it with a new, sterile valve to continue the procedure without significant delay. However, if the debris causes the valve to seize in a manner that prevents its removal, or if the design is integrated such that it cannot be swapped in the field, the situation becomes much more serious. In these instances, the entire endoscope must be withdrawn from service and sent to a specialized facility for professional repair, as the instrument is no longer functional or safe for clinical use.

The present invention at least seeks to address issues of these problems, or at least to provide an alternative to the public.

### SUMMARY OF INVENTION

The first aspect of the present invention is related to a suction valve, adaptable to position in a port of an endoscope for assisting a cleaning procedure for said endoscope, comprising: a main body, comprising an internal cavity; a shaft, comprising a tubular structure being made of a first material selected from thermosetting material, resilient material, or a combination thereof and comprising a first opening on the tubular wall of said tubular structure and a second opening at the bottom of said tubular structure, wherein said first opening and said second opening being fluidic communicable defining an internal channel inside said tubular structure; a button; a resilient member, positioned between said button and said main body, said main body, said shaft, said button, and said resilient member being assembled in a way such that said button together with said shaft being configured to reciprocally move between a first position where said first opening of said shaft being at least partially aligned with a first opening of said port and a second position where said first opening of said shaft being misaligned with said first opening of said port.

In some embodiments, said first material being selected from the following:
thermoplastic elastomer (TPE);
polypropylene (PP), polyphenylene ether (PPE), high-density polyethylene (HDPE), rubber, acrylonitrile butadiene styrene (ABS), polyvinyl chloride (PVC);
silicone; or
a combination thereof.

In some embodiments, the hardness of said first material being in a range of 30 Shore-A to 95 Shore-A.

In some embodiments, said first material being selected from TPE, or silicone, Polyvinyl Chloride (PVC), or a combination thereof.

In some embodiments, the thickness of the tubular structure of said tubular structure ≥0.5 mm.

In some embodiments, the thickness of the tubular wall of said tubular structure ≥1 mm.

In some embodiments, said first material being selected from TPE, or silicone, Polyvinyl Chloride (PVC), or a combination thereof; and the thickness of the tubular wall of said tubular structure ≥0.5 mm.

In some embodiments, the hardness of said first material ≤70 Shore-D.

In some embodiments, said first material being selected from polypropylene (PP), polyphenylene ether (PPE), high-density polyethylene (HDPE), rubber, acrylonitrile butadiene styrene (ABS), polyvinyl chloride (PVC), or a combination thereof.

In some embodiments, the thickness of the tubular wall of said tubular structure ≤0.5 mm.

In some embodiments, said first material being selected from polypropylene (PP), polyphenylene ether (PPE), high-density polyethylene (HDPE), rubber, acrylonitrile butadiene styrene (ABS), polyvinyl chloride (PVC), or a combination thereof; and the thickness of the tubular wall of said tubular structure ≤0.5 mm.

In some embodiments, said internal channel comprising a first profile defined at least by said first opening of said shaft and a second profile defined at least by said second opening of said shaft.

In some embodiments, said shaft comprising
an upper portion comprising a coupling structure;
a lower portion comprising a coupling structure connecting said tubular structure; and

wherein said coupling structure of said upper portion coupling with said coupling structure of said lower portion, whereby said upper portion of said shaft removably assembling to said lower portion of said shaft.

In some embodiments, said lower portion and said tubular structure being manufactured as a single piece.

In some embodiments, said coupling structure of said lower portion, said lower portion, and said tubular structure being manufactured as a single piece.

In some embodiments, said upper portion further comprising an engagement structure engageable with an engagement structure of said button, whereby said button removably assembling to said upper portion of said shaft.

In some embodiments, said upper portion further comprising an engagement structure engageable with an engagement structure of said button, whereby said button removably assembling to said upper portion of said shaft.

### LIST OF DRAWINGS

Some embodiments of the present invention will now be explained, with reference to the accompanied drawings, in which:

FIG. 1A is a perspective view of a first embodiment of a suction valve of the present invention in an unpressed condition;

FIG. 1B is a perspective view of the first embodiment of the suction valve of the present invention in a pressed condition;

FIG. 2A is a sectional view of the first embodiment of the suction valve of the present invention in an unpressed condition;

FIG. 2B is a sectional view of the first embodiment of the suction valve of the present invention in a pressed condition;

FIG. 3A is a perspective view of a second embodiment of a suction valve of the present invention in an unpressed condition;

FIG. 3B is a perspective view of the second embodiment of the suction valve of the present invention in a pressed condition;

FIG. 4A is a sectional view of the second embodiment of the suction valve of the present invention in an unpressed condition; and

FIG. 4B is a sectional view of the second embodiment of the suction valve of the present invention in a pressed condition.

### DETAILED DESCRIPTION OF PREFERRED EMBODIMENTS

The present invention is now presented by way of examples with reference to the figures in the following paragraphs. Objects, features, and aspects of the present disclosure are disclosed in or are apparent from the following description. It should be understood by one of ordinary skilled in the art that the following description is a description of exemplary embodiments only, and is not intended as limiting the broader aspects of the present disclosure, which broader aspects are embodied in the exemplary constructions.

It should be noted that, unless otherwise defined, the technical terms or scientific terms used in the embodiments of the present invention shall have the usual meanings understood by person with ordinary skills in the art to which the present invention belongs. “First”, “second” and similar expression used in the embodiments of the present invention do not indicate any order, quantity or importance, but are only used to distinguish different components. “Front”, “rear”, “left”, “right”, “upper”, and “lower” and other terms indicating orientation or similar terms are only described for the exemplary relative positional relationship shown in the drawings to facilitate the understanding. It does not limit the disclosed components in the present invention can only follow this specific relative positional relationship.

For brevity's sake, the operation of an endoscope, the connection means of the endoscope to a water source, air source, and suction source are not described and explained in this specification, as this information constitute the state of art.

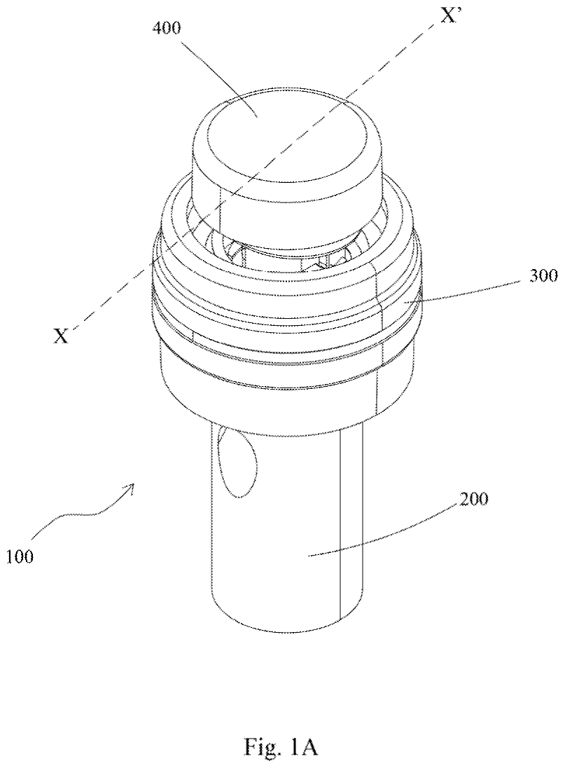

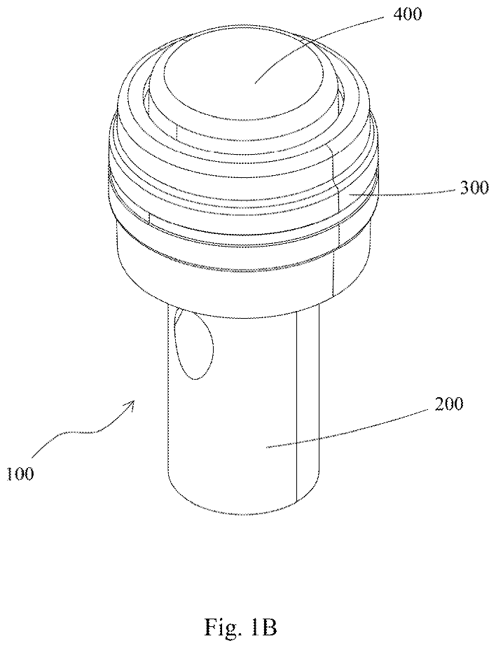

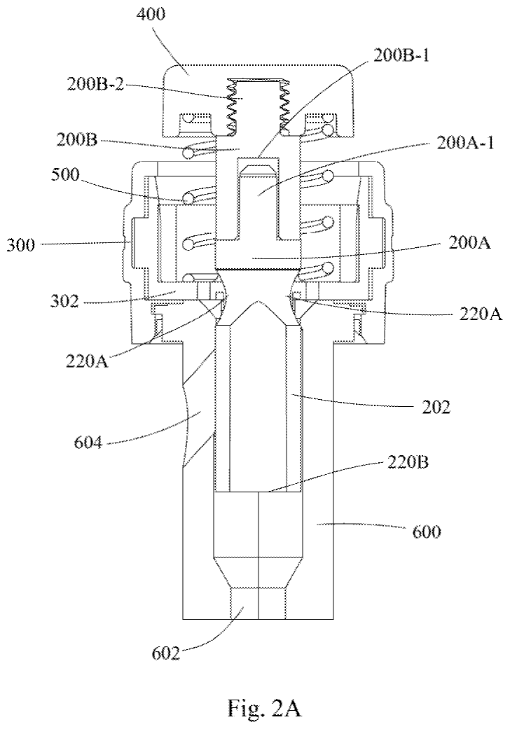

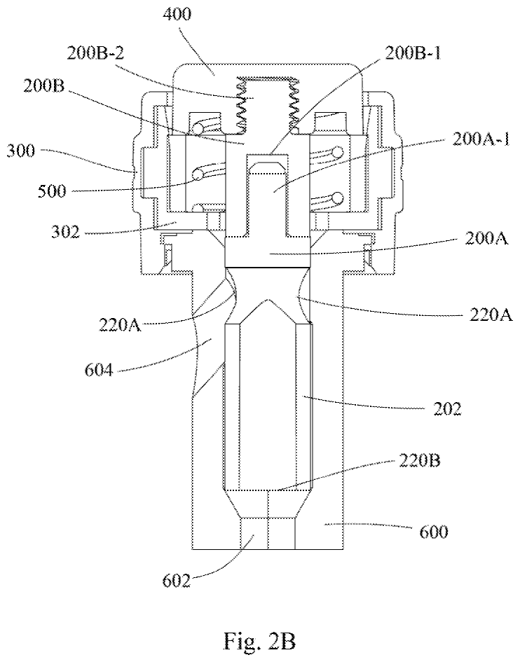

FIG. 1A is a perspective view of a first embodiment of a suction valve 100 of the present invention in an unpressed condition. FIG. 1B is a perspective view of the suction valve 100 in a pressed condition. FIG. 2A is a sectional view, along dotted line XX′ in FIG. 1A, of the suction valve 100 in the unpressed condition. FIG. 2B is a second sectional view, along dotted line XX′ in FIG. 1A, of the suction valve 100 in the pressed condition.

FIGS. 1A-1B and 2A-2B show that the suction valve 100 comprises a shaft 200, a main body 300, and a button 400. The shaft comprises a lower portion 200A and an upper portion 200B, which are configured to be removably coupled to each other, for example, via snug fit, snap fit, gluing, and/or tight fit. It shall be important to note that the listed methods for coupling are examples only; those skilled in the art could configure it according to actual needs. Advantageously, the hardness of the upper portion 200B is higher than that of the lower portion 200A.

The lower portion 200A and the upper portion 200B are each provided with a coupling structure to enable interconnection. Referring to FIGS. 2A and 2B, the coupling structure of the lower portion 200A is configured as a male coupler 200A-1, while the coupling structure of the upper portion 200B is configured as a female coupler 200B-1. It should be noted that these coupling structures are exemplary; those skilled in the art may configure them according to specific requirements. For instance, the lower portion 200A may comprise a female coupler while the upper portion 200B comprises a male coupler. The male coupler 200A-1 and female coupler 200B-1 are removably coupled, such as via a snug fit. However, a snug fit is only one example, and other suitable coupling methods may be employed as needed. Advantageously, the hardness of the coupling structure of the upper portion 200B is higher than that of the coupling structure of the lower portion 200A.

The upper portion 200B further comprises an engagement structure 200B-2, exemplified here as a threaded screw. The engagement structure 200B-2 is configured to mate with an engagement structure 400-1 of the button 400 (also illustratively shown as a threaded screw) via screwing, such that the button 400 and the upper portion 200B are removably engaged with each other. In other words, the button 400 and the upper portion 200B are assembled by interlocking their respective engagement structures 400-1 and 200B-2. It should be noted that the configurations of 400-1 and 200B-2 shown in the figures are examples only; those skilled in the art may configure them according to specific requirements.

The lower portion 200A is constructed to comprise the coupling structure and a tubular structure connecting the coupling structure. As illustrated in FIGS. 1A-1B and 2A-2B., the tubular structure of the lower portion 200A connects with the male coupler 200A-1.

Integral to the function of the tubular structure is a T-shaped internal channel 220, which serves as a multifunctional fluid conduit. In this specification, “fluid” may refer to liquid or gas. Although the internal channel 220 is illustrated as T-shaped in the figures, this is for exemplary purposes only. The essence of the internal channel is to provide a passage with at least one fluid inlet and at least one fluid outlet. The internal channel is configured to function, for example, as a controlled passage for fluids, facilitating the directed flow of cleaning fluids. In some embodiments, the internal channel functions as a suction vacuum tube or a suction connecting tube in endoscopic procedures. As shown in FIGS. 2A-2B, the internal channel 220 comprises a horizontal portion—defined by a hollow channel having a pair of opposite openings 220A on the tubular wall 202—and a vertical portion, which are in fluidic communication. The vertical portion extends downward from the midpoint of the horizontal portion to an opening 220B at the bottom of the shaft 200 (or one end of the tubular structure of the lower portion 200A). It should be noted that the internal channel does not necessarily have to be T-shaped. The scope of the internal channel encompasses any structure where a horizontal profile and a vertical profile are in fluidic communication. As long as an internal channel having a horizontal profile and a vertically profile fluidically communicable with each other, in which the horizontal profile is realized by a first opening provided on the length side of the tubular structure of the shaft extending into the tubular structure, while the vertical profile is realized by a second opening provided on the bottom of the tubular structure of the shaft extending upwards forming the vertical profile meeting the horizontal profile enabling the fluidic communication, it also falls into the gist of the internal channel. In some embodiments, the shape of the internal channel can be Y-shaped, inverted A-shaped, or any shape. In some embodiments, the internal channel has three, four, five or even more openings according to actual needs.

A resilient member 500 is positioned between the button 400 and the main body 300, in particular between a recess provided at the underside of the button 400 and a limiting structure 302 of the main body 300.

Based on the above description, the shaft 200 and the button 400 are operably assembled. A resilient member 500, positioned between the button 400 and the main body 300, facilitates the positioning of the button 400 and the shaft 200. When a user actuates the button 400 by pressing against the resilient member 500, the button 400 and the shaft 200 are moved from a first position to a second position (as depicted in FIG. 2B), where a substantial portion of the shaft 200 is received within the internal cavity of the port 600. Upon releasing the button 400, the compressed resilient member 500 returns to its original state, moving the button 400 and the shaft 200 from the second position back to the first position (as depicted in FIG. 2A). In this first position, the portion of the shaft 200 received in the internal cavity of the port 600 is less than that in the second position.

The shaft 200 is at least partially housed inside an accommodation cavity defined by the internal cavity of the main body 300 and the internal cavity of the port 600. Advantageously, the depth of the internal cavity of the port 600 is greater than the length of the tubular structure of the shaft 200, such that the tubular structure is movable within the internal cavity of the port 600. Specifically, with the aid of the resilient member 500 disposed between the button 400 and the main body 300, the tubular structure of the shaft 200 is configured to move reciprocally within the internal cavity of the port 600.

FIGS. 2A-2B show that the suction valve 100 is removably mounted into a port 600 forming part of an endoscope. The port 600 is provided with a first opening 602 and a second opening 604. As shown, the vertical portion of the internal channel 220 aligns with the first opening 602 of the port 600 regardless of whether the suction valve 100 is in an unpressed state or a pressed state.

FIG. 2A shows that when the suction valve 100 is in the unpressed state, the opening 220A of the horizontal portion is offset from or is misaligned with the second opening 604 of the port 600. Conversely, FIG. 2B shows that when the suction valve 100 is in the pressed state, the opening 220A is at least partially aligned with the second opening 604.

Facilitated by the resilient member 500, the tubular structure of the shaft 200 reciprocates within the port 600 between a first position, where the opening 220A is offset from or is misaligned with the second opening 604, and a second position, where the opening 220A is at least partially aligned with the second opening 604. Referring to FIGS. 2A and 2B, the opening 220B in the first position is further from the first opening 602 than it is in the second position.

In endoscopic procedures, the suction valve 100 functions as a manual mechanical gate that regulates the application of negative pressure from an external vacuum source (not shown in figures) to the endoscope's internal channels (not shown in figures). The suction valve 100 is typically located on the control section of the endoscope.

The suction valve 100 operates through a resilient member-loaded button which operably controls the pressed condition and the unpressed condition of the suction valve. The lower portion 200A of the shaft 200 is operably housed within the internal cavity of the port 600. In its unpressed condition, the tubular structure is held in an “up” position, effectively blocking the path between the suction source and the internal channel 220 of the shaft 200. During this time, the suction system remains under constant negative pressure, but the sealing effect provided by the material of the tubular structure of the shaft 200 prevents this vacuum from reaching the distal tip of the endoscope.

When the medical personnel press the suction valve, the tubular structure of the shaft 200 moves downward into the internal cavity of the port 600, aligning the opening 220A of the horizontal profile of the internal channel 220 at least partially overlapped with the second opening 604 of the port 600. This alignment creates a continuous pathway that allows the vacuum to pull fluids, air, or debris from the distal tip of the endoscope, through the biopsy/suction channel of the insertion tube of the endoscope, and into an external collection canister. Once the medical personnel release the button 400, the resilient member 500 automatically returns the tubular structure of the shaft 200 to its original position, immediately severing the vacuum connection and stopping the suction process.

The present invention relates to an improved design for a suction valve utilized in endoscopic procedures, with a particular focus on the material attributes of the shaft 200. This shaft 200 is fabricated from thermosetting material, resilient material, or a combination thereof, which may include, but is not limited to thermoplastic elastomer (TPE), polypropylene (PP), polyphenylene ether (PPE), high-density polyethylene (HDPE), rubber, Acrylonitrile Butadiene Styrene (ABS), Polyvinyl Chloride (PVC), silicone, or a combination thereof.

In some embodiments, the thermosetting material, resilient material, or a combination thereof may include TPE, silicone, Polyvinyl Chloride (PVC), or a combination thereof. The hardness of the TPE and the silicone and the Polyvinyl Chloride (PVC) is carefully controlled to fall within a range of 30 Shore-A to 95 Shore-A, ensuring that the shaft possesses the necessary flexibility to deform under stress while retaining sufficient resilience to return to its original shape.

In some embodiments, the thickness of the tubular wall of said tubular structure can be configured to ≥0.5 mm. In some embodiments, the thickness of the tubular wall of said tubular structure can be configured to ≥1 mm. In the present disclosure, “thickness of the tubular wall of said tubular structure” refers to the thickness of tubing, that is the dimension of the material between the inner and outer surfaces, calculated by taking the difference between the outside diameter and the inside diameter and dividing that value by two.

A significant advantage of this invention is realized during endoscopic procedures when debris, such as seeds or bone fragments, inadvertently block the opening of the port or the opening of the shaft. In such instances, the shaft of the suction valve deforms temporarily around the debris, demonstrating a compliance that allows the valve to continue its stroke or revert to its original position without becoming permanently jammed. By yielding to the obstruction rather than locking against it, the material used in the present invention, as disclosed in this specification, ensures that minor ingestions of debris do not escalate into clinical emergencies, thereby obviating the need for immediate removal of the endoscope from the patient. This feature enhances the safety and reliability of endoscopic interventions, preventing procedural interruptions and reducing the risk of complications, ultimately contributing to improved patient outcomes.

With regard to the embodiments in FIGS. 1A-1B and 2A-2B, it shall be important to note that when the hardness of the material used in the present invention resides in a range between 30 Shore-A and 95 Shore-A, there is no limitation on the thickness of the tubular wall which can provide the deformable effect around the debris inadvertently blocking the opening of the port or the opening of the shaft.

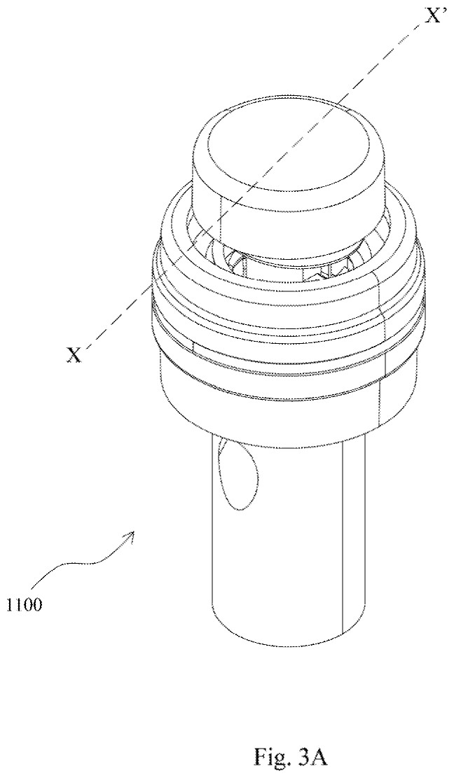

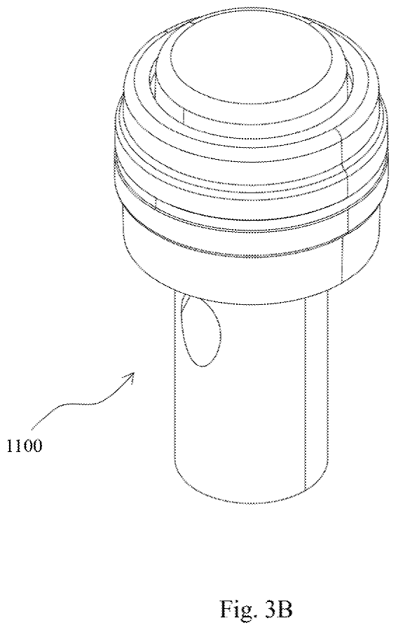

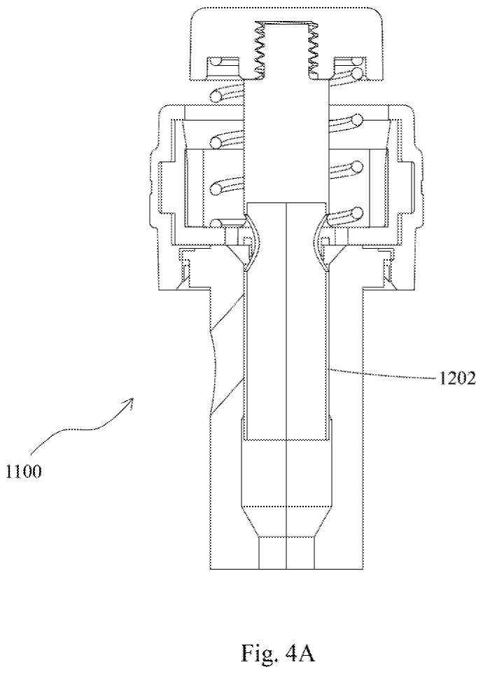

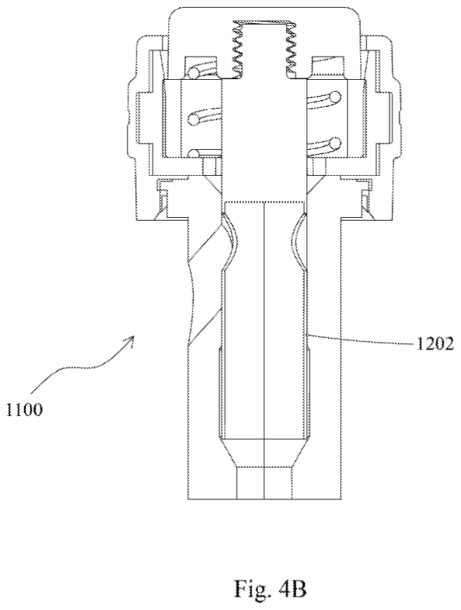

FIG. 3A is a perspective view of a second embodiment of a suction valve 1100 of the present invention in an unpressed condition. FIG. 3B is a perspective view of the suction valve 1100 in a pressed condition. FIG. 4A is a sectional view, along dotted line XX′ in FIG. 3A, of the suction valve 1100 in the unpressed condition. FIG. 4B is a second sectional view, along dotted line XX′ in FIG. 3A, of the suction valve 1100 in the pressed condition.

The suction valve 1100 is generally similar to the suction valve 100. For sake of brevity and clarity, only major differences are described. One main difference is that, as clearly shown in for example FIGS. 4A-4B, the thickness of the tubular wall 1202 can be configured thinner than that of the tubular wall 202. In addition to the description for FIGS. 1A-1B and 2A-2B, the present invention also relates to a particular focus on the structural attributes of the shaft 1100, i.e., the thin tubular wall 1202. Advantageously, the thickness of the tubular wall 1202 ≤0.5 millimeters. In some embodiments, the thickness of the tubular wall 1202 is in a range between 0.1 and 0.5 millimeters.

In some embodiments, the shaft in suction valve 1100 can be integrally formed, constituting a difference between the suction valve 100 and the suction valve 1100. On the contrary, the shaft in the suction valve 100 comprises a lower portion 200A and an upper portion 200B.

In some embodiments, the thermosetting material, resilient material, or a combination thereof used in this embodiment for the tubular wall 1202 is selected from polypropylene (PP), polyphenylene ether (PPE), high-density polyethylene (HDPE), rubber, acrylonitrile butadiene styrene (ABS), polyvinyl chloride (PVC), or a combination thereof.

In some embodiments, the hardness of material used for the tubular wall 1202 ≤70 Shore-D.

By citing Polyvinyl Chloride (PVC), the description above highlights the material's unique versatility, as it can be engineered to span a vast spectrum of textures—from highly flexible, rubber-like forms (30 to 95 Shore-A) to rigid, structural forms (up to 70 Shore-D). This example implies that a single material like PVC can function as both a “soft” and “hard” component depending on its formulation, naming the material itself does not provide enough technical specificity. Therefore, the success of the invention relies on the mechanical resistance or tactile feedback of the component rather than its molecular makeup. One of the essences of the present invention is to prioritize physical performance over chemical composition within the context of the invention.

The present invention enables the transition from a rigid metal shaft to one preferably constructed from thermosetting material and/or resilient material-including but not limited to thermoplastic elastomer (TPE), polypropylene (PP), polyphenylene ether (PPE), high-density polyethylene (HDPE), rubber, Acrylonitrile Butadiene Styrene (ABS), Polyvinyl Chloride (PVC), silicone, or a combination thereof-which offers a transformative advantage in mitigating the risk of mechanical failure during a procedure. While a metal shaft is prone to immediate seizing when a hard particle becomes trapped in its narrow clearance, a resilient shaft or a deformable shaft due to the use of the thermosetting material, resilient material, or a combination thereof possesses the inherent elasticity to deform momentarily around the debris. This “compliance” allows the valve to continue its stroke or return to its original position despite the presence of seeds or bone fragments, effectively preventing the valve from becoming permanently jammed. By yielding to the obstruction rather than locking against it, the material used in the present invention ensures that a minor ingestion of debris does not escalate into a clinical emergency or require the immediate removal of the endoscope from the patient.

Furthermore, the use of resilient shaft or deformable shaft drastically reduces the long-term maintenance burden by eliminating the abrasive wearing inherent in metal-on-metal or metal-on-plastic interfaces. Because the shaft of the present invention is composed of a material softer than the internal structure of the endoscope handle, it acts as a protective component rather than an abrasive one. Instead of scoring or galling the internal cylinder walls, the shaft of the present invention absorbs the friction of repeated cycles, preserving the integrity of the endoscope's expensive internal housing. This fundamental shift in material properties means that the “wear and tear” is concentrated on the replaceable valve unit rather than the permanent instrument, significantly extending the operational lifespan of the endoscope and reducing the need for costly factory repairs.

Additionally, the sealing capability of the shaft of the present invention is superior in a contaminated environment compared to its rigid counterparts. The shaft of the present invention, which is preferably made of thermosetting material, resilient material, or a combination thereof, can function as its own dynamic seal, conforming more tightly to the internal contours of the valve chamber even as those surfaces age. This ensures a consistent vacuum seal and prevents the bypass of fluids into the control handle, which is a common failure point in metal designs. By combining the ability to bypass physical obstructions with a non-destructive relationship to the endoscope's internal hardware, the present invention provides a cost-effective, and patient-safe solution for modern endoscopic surgery.

Apart from the above, the transition from a rigid metal shaft to thermosetting material, resilient material, or a combination thereof provides significant industrial and clinical benefits, particularly regarding manufacturing efficiency, cost structures, and hygiene standards.

From a production standpoint, switching to a resilient shaft or a deformable shaft, such as polymer shaft, allows for the use of high-volume injection molding, which is inherently more efficient than the precision CNC machining required for stainless steel shafts. Metal components often require multiple secondary processes—such as grinding, polishing, and passivating—to achieve the necessary surface finish and corrosion resistance. In contrast, the shafts of the present invention can be produced in a single-step process that creates complex geometries and integrated seals (like O-rings) as part of a single mold, significantly reducing assembly time and labor requirements. This leads to faster manufacturing cycles and a much shorter time-to-market for high-demand medical supplies.

The raw material cost of medical-grade polymers is substantially lower than that of specialty alloys. While the initial investment in high-quality molds can be significant, the per-unit cost drops dramatically during large-scale production runs, making the valve of the present invention a far more economical alternative. These savings translate directly into lower operational costs for healthcare facilities. Because the resilient shaft or deformable shaft is designed for affordability, it enables a single-use (disposable) model that eliminates the “hidden” expenses associated with reusable equipment, such as specialized cleaning detergents, high-level disinfection (HLD) systems, and the intensive labor required for manual reprocessing. Hospitals can also avoid the high capital depreciation and expensive long-term repair contracts typically required to maintain metal-based endoscopic instruments.

The most critical advantage of a resilient and/or deformable, single-use suction valve is the elimination of cross-contamination risks. Reusable metal valves are notorious for harboring residual biofilms and microscopic debris in their recessed channels, which can survive even stringent sterilization protocols. By providing a new, sterile valve for every procedure, the present invention removes the risk of hospital-acquired infections and ensures that the mechanical performance—such as suction responsiveness and tactile feedback—is consistently optimal and never degraded by prior use or wear. This “one-and-done” approach simplifies clinical workflows, as staff no longer need to track and validate the sterilization history of individual valve sets.

The above-described shall not be interpreted to be restricted by the examples or figures only. It is to be expressly understood, however, that such modifications and adaptations are within the scope of invention in this aspect. For instance, features illustrated or described as part of one embodiment can be used on another embodiment to yield a still further embodiment. Thus, it is intended that the present disclosure cover such modifications and variations and their equivalents. Any numerical value disclosed herein shall be construed to encompass its technical equivalents that perform substantially the same function in substantially the same way to achieve substantially the same result as the recited value.

What is claimed is:

1. A suction valve, adaptable to position in a port of an endoscope for assisting a cleaning procedure for said endoscope, comprising:
a main body, comprising an internal cavity;
a shaft, comprising a tubular structure being made of a first material selected from thermosetting material, resilient material, or a combination thereof and comprising a first opening on the tubular wall of said tubular structure and a second opening at the bottom of said tubular structure, wherein said first opening and said second opening being fluidic communicable defining an internal channel inside said tubular structure;
a button;
a resilient member, positioned between said button and said main body,
said main body, said shaft, said button, and said resilient member being assembled in a way such that said button together with said shaft being configured to reciprocally move between a first position where said first opening of said shaft being at least partially aligned with a first opening of said port and a second position where said first opening of said shaft being misaligned with said first opening of said port.

2. The suction valve according to claim 1, characterized in that, said first material being selected from the following:
thermoplastic elastomer (TPE);
polypropylene (PP), polyphenylene ether (PPE), high-density polyethylene (HDPE),
rubber, acrylonitrile butadiene styrene (ABS), polyvinyl chloride (PVC);
silicone; or
a combination thereof.

3. The suction valve according to claim 1, characterized in that, the hardness of said first material being in a range of 30 Shore-A to 95 Shore-A.

4. The suction valve according to claim 3, characterized in that, said first material being selected from TPE, or silicone, Polyvinyl Chloride (PVC), or a combination thereof.

5. The suction valve according to claim 3, characterized in that, the thickness of the tubular structure of said tubular structure ≥0.5 mm.

6. The suction valve according to claim 3, characterized in that, the thickness of the tubular wall of said tubular structure ≥1 mm.

7. The suction valve according to claim 3, characterized in that, said first material being selected from TPE, or silicone, Polyvinyl Chloride (PVC), or a combination thereof; and the thickness of the tubular wall of said tubular structure ≥0.5 mm.

8. The suction valve according to claim 1, characterized in that, the hardness of said first material ≤70 Shore-D.

9. The suction valve according to claim 8, characterized in that, said first material being selected from polypropylene (PP), polyphenylene ether (PPE), high-density polyethylene (HDPE), rubber, acrylonitrile butadiene styrene (ABS), polyvinyl chloride (PVC), or a combination thereof.

10. The suction valve according to claim 8, characterized in that, the thickness of the tubular wall of said tubular structure ≤0.5 mm.

11. The suction valve according to claim 8, characterized in that, said first material being selected from polypropylene (PP), polyphenylene ether (PPE), high-density polyethylene (HDPE), rubber, acrylonitrile butadiene styrene (ABS), polyvinyl chloride (PVC), or a combination thereof; and the thickness of the tubular wall of said tubular structure ≤0.5 mm.

12. The suction valve according to claim 2, characterized in that, said internal channel comprising a first profile defined at least by said first opening of said shaft and a second profile defined at least by said second opening of said shaft.

13. The suction valve according to claim 12, characterized in that, said shaft comprising
an upper portion comprising a coupling structure;
a lower portion comprising a coupling structure connecting said tubular structure; and
wherein said coupling structure of said upper portion coupling with said coupling structure of said lower portion, whereby said upper portion of said shaft removably assembling to said lower portion of said shaft.

14. The suction valve according to claim 13, characterized in that, said lower portion and said tubular structure being manufactured as a single piece.

15. The suction valve according to claim 13, characterized in that, said coupling structure of said lower portion, said lower portion, and said tubular structure being manufactured as a single piece.

16. The suction valve according to claim 14, characterized in that, said upper portion further comprising an engagement structure engageable with an engagement structure of said button, whereby said button removably assembling to said upper portion of said shaft.

17. The suction valve according to claim 15, characterized in that, said upper portion further comprising an engagement structure engageable with an engagement structure of said button, whereby said button removably assembling to said upper portion of said shaft.
